(19) United States
(12) Patent Application Publication
(10) Pub. No.: US 2008/0305045 A1
(43) Pub. Date: Dec. 11, 2008

(54) Methods of synthesis of non-toxic multifunctional nanoparticles and applications

(76) Inventors: Prabhakaran Kuniyil, Fremont, CA (US); Geetha Prabhakaran, Fremont, CA (US)

Correspondence Address: Katherine N. Addison Esq., 501 Marie Avenue, Los Angeles, CA, 90042-1305, US

(21) Appl. No.: 12/135,206

(22) Filed: Jun. 9, 2008

Related U.S. Application Data
(60) Provisional application No. 60/942,501, filed on Jun. 7, 2007.

(51) Int. Cl. A61K 49/00; H01F 1/14; F21V 9/04

(52) U.S. Cl. 424/9.1; 252/587; 252/62.51 R; 252/62.55

(57) ABSTRACT

The present invention involves multifunctional nanoparticle dispersions and methods for making them using sol-gel chemistry, doping, and sonication. These methods avoid the high thermal budget processes of the reference art. The dispersions can accommodate greater concentrations of nanoparticles, dopants, and ions than has previously been possible since these components can be added during synthesis. The unique optical, magnetic, luminescent, metallic, insulating, semi-conducting, and/or conducting properties of these particles can be utilized to enhance photovoltaic cells, portable electronic devices, and biomedical techniques among other applications.

### BACKGROUND OF THE INVENTION

1. Field of the Invention

The invention pertains to the art of nanotechnology. More specifically, the invention provides nanoparticles, nanostructures, and fabrication methods with improved tunability for specialized and diverse applications. Most specifically, the invention provides: (i) non-toxic nanomaterials in which large amounts of dopant ions can be incorporated through improved solubilities of nanoparticles in solution; and (ii) faster, large-scale processes for nanomaterial substrate design using superficial chemical reactivity differences and gas phase reactions.

2. Description of the Related Art

Various other devices, systems, and methods have been presented dealing with nanoparticle synthesis and applications in the fields of: biotechnology, securities technology, medicine, energy, optics, and electronics (among others).

There have been significant efforts to develop nanoparticles that exhibit interesting functionalities. However, many of the nanoparticles developed so far suffer from the problem of potential toxicity, due to elements such as the heavy metals Cd, Hg, etc. Thus, the use of these nanomaterials for real world applications involving human interaction has been limited, in part due to toxicity risks. This points to a need in the art for the development of non-toxic nanoparticles exhibiting novel functional properties.

One of the most important applications for nanomaterials is in natural energy generation technology, including solar cells. Conventional solar cells are limited in their performance due to a variety of reasons. First, the light conversion aspect is restricted to the acceptance of intense natural “solar” energy from the sun. Traditional solar cells and panels are not effectively stimulated by artificial indoor ambient light and are also not very effective on cloudy days. Therefore, solar cells are not very reliable in many regions where the weather pattern does not guarantee sunshine. Even in sunny climates solar panels must be positioned at special angles (i.e. 22°) and are not as efficient as they should be with their harvesting potential restricted to the peak sun hours of the day (i.e. 1-7 pm).

U.S. Pat. No. (hereinafter USP) 7,306,823 (by Sagar, et al. and assigned to Nanosolar, Inc.) discloses “Coated nanoparticles and quantum dots for solution-based fabrication of photovoltaic cells”. However, its teachings are distinguishable from the present invention because it is directed to non-silicon based solar cells by printing or web coating solutions of copper (Cu), indium (In), gallium (Ga), selenium (Se), and/or sulfur (S) onto flexible substrates. These are referred to as CIGS cells. In contrast, the techniques of the present invention are designed to work with existing silicon-based solar cells. U.S. Pat. No. '823 recognizes drawbacks in the existing methods of solar cell fabrication but teaches away from the techniques employed in the present invention used to overcome those drawbacks. U.S. Pat. No. '823 blames some of the disadvantages of conventional solar cell fabrication on its use of oxides and specifically declares the need for “a non-oxide, nanoparticle based precursor material.” (See 2:42-44.) U.S. Pat. No. '823 claims several techniques for forming core nanoparticles: “evaporation-condensation, electroexplosion of wire, organometallic synthesis, metal salt reduction, and/or a combination of high temperature decomposition of a metal carbonyl precursor and the reduction of a metal salt in the presence of surfactants, and/or combinations of these techniques” (see claim 26). Sol-gel chemistry and sonication are not included among this list. Nanoparticles can be coated and the preferred coating method taught by U.S. Pat. No. '823 appears to be atomic layer deposition (see claim 27). The present invention provides alternative methods for coating better suited to large-scale production processes.

With regard to the formulation of nanostructures on substrates for application in electronic devices, prior art techniques (i.e. using scanning tunneling microscopy (STM) or atomic force microscopy (ATM) tips) for nano-scale manipulation and design are impractical for large-scale industrial manufacturing applications. The present invention provides commercially practical methods for spatial selectivity and controlled nanostructure surface reactions (i.e. including gaseous phase reactions) that are capable of forming various functional units on an entire wafer surface in a single step. Gaseous surface reactions can be used to differentiate a smooth substrate surface by engineering dissimilarities in surface atoms through mechanisms including: facet formation, step bunching, step ordering, stress/strain distribution, etc. The techniques of the present invention can be used to ensure uniformity in size and spatial distribution of the nanoparticles for more densely packed layers with superior optical and magnetic properties.

U.S. Pat. No. 7,083,104 (by Empedocles, et al. and assigned to Nanosys, Inc.) discloses “Applications of nano-enabled large area macroelectronic substrates incorporating nanowires and nanowire composites”. However, it emphasizes nanowires rather than nanoparticles and relies primarily upon radio frequency energy rather than ultraviolet, infrared, and visible light. As its title suggests U.S. Pat. No. '104 deals primarily with integration of nanomaterials (nanowires) into a macroelectronic substrate. The nanowires form a thin film electrically interconnected to a source and drain. The nanowire film functions as a transistor in a switch used to adjust the phase of a tunable element in a beam-steering array to redirect an electromagnetic signal generated by an antenna. It does not address manipulation of the chemical synthesis of nanomaterials by using sonication during doping. It also does not refer to the “bottom-up” or “plug-and-play” approach to fabrication based on the production of stand-alone nanostructures nor to nanoparticle-semiconductor hybrid structures ready-made with built-in electrical functionalities.

U.S. Pat. No. 6,576,355 (by Yadav, et al. and assigned to NanoProducts Corporation) discloses “Nanotechnology for electronic and opto-electronic devices” that uses only non-stoichiometric, non-equilibrium, crystalline nanomaterial. The nanotechnology of the present invention also applies to stoichiometric, equilibrium, and non-crystalline (i.e. amorphous) nanomaterials. U.S. Pat. No. '355 is also limited by the domain size of the nanomaterial which is explicitly “confined to a dimension less than the mean free path of electrons in the material composition” (see independent claim 1). It does not address increasing the dopant holding capacity of nanomaterials via dynamic infusion into a liquid medium to form a concentrated dispersion. Two illustrations in U.S. Pat. No. '355 refer to adding dopants after sonication of the nanomaterial mixture. (See 4:28-29 and 21:25-32.) Nor does U.S. Pat. No. '355 refer to producing nanomaterials from isoproxides using sol-gel chemistry and re-sonification.

U.S. Pat. No. 7,329,638 (by Yang, et al. and assigned to the Regents of the University of Michigan) discloses “Drug delivery compositions”. The nanomaterials of the present invention may also be applied for precision drug delivery. However, U.S. Pat. No. '638 is limited to multicomponent compositions with a particular template and focuses on translocating molecules across membranes. More specifically, the delivery compositions of U.S. Pat. No. '638 must have:

(i) a positively charged cationic molecule with at least a portion of a protein transduction domain with at least a portion of a TAT protein or a Low Molecular Weight Protamine (LMWP) protein;
(ii) a magnetic nanoparticle associated with the positively charged cationic molecule;
(iii) a small molecule drug as a therapeutic agent; and
(iv) a molecular recognition element with a polypeptide that is VEGF or an anti-CD20 antibody. The nanomaterial transporters of the present invention are not structurally limited and are able to transport within bodily lumens such as vessels, channels, etc. in which no membranes need to be crossed.

U.S. Pat. No. 7,296,576 (by Ait-Haddou, et al. and assigned to Zyvex Materials Performance, LLC) discloses “Polymers for enhanced solubility of nanomaterials, compositions and methods therefore” focusing on poly(aryleneethynylene), poly(ferrocenylaryleneethynylene) and poly(ferrocenylethynylene) polymers each having at least one functional group for solubilizing nanomaterial. The methods of the present invention can solubilize nanomaterials together with dopants sufficiently for improved dopant incorporation using simple and readily available solvents such as ethanol. This avoids the extra time and/or expense of making special polymers to solubilize the nanomaterial. U.S. Pat. No. '576 refers only to solubilizing nanomaterials but not to doping them and thus it is not clear that the solubilization methods taught therein would facilitate improved dopant holding capacities. U.S. Pat. No. '576 also does not discuss the formation or solubilization of isopropoxide derived nanomaterials.

### BRIEF SUMMARY OF THE INVENTION

The application possibilities for functional nanoparticles (luminescent, optical, magnetic, metallic, semiconducting, insulating, etc.) is crucially dependent on (1) how well they can be dispersed in ordinary solvents and (2) control of the structure and properties. The present invention presents a method to synthesize non-toxic, ultrafine, spherical particles of diameter 5-10 nm which are brightly luminescent and magnetically active by a unique combination of sol-gel techniques and sonochemistry by the decomposition of precursor molecules. The key idea involves sonication aided doping of the host lattice while the host lattice is being formed. This requires the co-presence of the dopant species and the host material in a solvent medium, which is being sonicated. We found that the sonicated mixture contains nanoparticles which possess the desired functional properties.

This approach of incorporation during formation has several advantages. First, it permits nanoparticles well dispersed in solvent to be obtained directly (i.e. by solubilizing the nanoparticles as they are formed). Ordinarily, solubilizing or solvating pre-formed nanoparticles is very difficult or virtually impossible to achieve. According to the present invention, nanoparticles including silicon dioxide (SiO₂), titanium oxide (TiO₂), and aluminum oxide (Al₂O₃) particles can be synthesized by decomposition of the respective isopropoxide sol-gels. They can be made functional by doping them with suitable dopants simultaneously with the formation of the host oxide material. We have performed this experiment using the dopants Eu, Fe, Zn, F, Cr, Co, Cu, Sn, Li, K, Mg, Mn, and Ce through their respective salt precursors. The doped nanoparticle dispersions and methods of the present invention thus provide several more possibilities and non-toxic or less toxic alternatives to the contemporary quantum dots being studied: i.e. CdSe, ZnSe, PbSe, PbS, ZnS, CdTe and CdHgTe. (See U.S. Published Application No. 2008/0066802 at paragraphs [0015] and [0024]; U.S. Published Application No. 2007/0194694 at paragraph [0012] and claim 20; and U.S. Pat. No. 6,884,478 at 4:23-32.) U.S. Published Application No. 2008/0066802 considers the quantum dot to be a preferred nanoparticle, explaining that dots having the same composition but different diameters can absorb and emit at different wavelengths and highlighting various heavy metal selenides (ZnSe, CdSe, and PbSe). (See [0052].) By tuning the concentration and composition of the doped nanoparticle dispersions of the present invention, non-toxic uniformly sized particles can absorb and emit in different regions of the spectrum. The methods of the present invention facilitate the production of a well-mixed substantially homogenous solution of non-toxic nanoparticles with a high concentration of functional dopants. Particle size uniformity preserves crystallinity and optical and magnetic properties. Non-toxic materials permit greater concentrations of photosensitive or magnetically active dopants to increase efficiency, luminescence and power without compromising safety, thereby expanding the range of applications.

Another advantage is that dispersions of solubilized, doped nanoparticles are generally cheaper to make than other nanomaterials produced by processes, like annealing and powderization, which requires high thermal budgets. In addition to the increased costs of long, high temperature formation processes, the decomposition products of some of the solvents or surfactants used therein have the potential to interfere with the desired optical properties of the nanocrystals. (See U.S. Pat. No. 7,193,098 at 1:30-46.)

Dispersions are an ideal medium for printing inks and toners. Nanoparticle dispersions can be made magnetic for security printing, authentication, and intellectual property (i.e. copyright) protection by incorporating magnetic ions as dopants. For example, the magnetic ions of elements including iron (Fe), chromium (Cr), and copper (Cu) can be used.

Dispersions of colored nanomaterials can be produced. Colored nanomaterials produce a particular color (wavelength) or multiple colors of light upon irradiation with suitable photons (i.e. photons of a particular wavelength). Depending upon the particular elements and dopants used the colors can be in the visible range or detectable only through ultraviolet (UV) or infrared (IR) sensitive viewers. Colored or multi-colored nanoparticles dispersed in solvents can be added to polymers to introduce luminescent properties to the polymers. These luminescent polymers can be used for laminating glass for a variety of applications. Our dispersions are entirely compatible with glass because the host material can be chosen to be silicon dioxide (SiO₂) from the precursor tetraethyl orthosilicate (TEOS).

Both “down conversion” and “up conversion” nanoparticle dispersions can be formed. “Down conversion” dispersions absorb UV radiation and emit white-light. These are well dispersed in ethanol and can be directly used for applications such as coating the surface of a silicon solar cell. Such dispersions can also be coated on windows to reduce glare and effectively convert natural UV-lighting to artificial, soft white lighting. “Up conversion” dispersions also emit visible light but form it by absorbing and converting IR rather than UV light. In one embodiment of an “up conversion” dispersion, silica is doped with rare earth ions (f block elements). “Up conversion” dispersions with nanoparticles distributed in a solvent can be obtained by sonicating TEOS with a rare earth salt such as lutetium nitrate (LuNO₃). “Up conversion” nano-dispersions that convert IR into visible light are particularly useful for expanding the solar cell market to less sunny areas and less sunny times. With the nanomaterials of the present invention investments in solar cell technology can be redeemed even during foul weather conditions (clouds, fog, smog, rain, snow) and even if the panels are placed on the wrong side of the roof.

Both “down conversion” and “up conversion” nanoparticle dispersion coatings can be applied to the surface of any solar cell to enhance its efficiency. Bifunctional coatings that can perform both types of conversion (UV and IR to visible), simultaneously or in a single layer, can also be used. These coatings provide potentially universal efficiency enhancement because their action is independent of the nature of the solar cell material. The efficaciousness of any type of solar cell can be sharpened in at least two ways. First, the effective amount of photons available for the solar cell for the conversion process (photons to electrons and then to current) can be significantly increased by converting the UV and IR components of the solar spectrum to visible light. Second, the nanoparticle coating has an anti-reflective property that effectively confines photons incident on the solar cell from being reflected off. One mechanism for achieving or enhancing the anti-reflective property of nanoparticle coatings, cells, and panels is through the use of nano-ring patterns. Nanometer-sized optically active rings show potential for special importance in the field of optical cavities and for use in resonators in which whispering gallery mode resonances are employed. (See Cai, M., Painter, O., Vahala, K. J. “Observation of critical coupling in a fiber taper to silica-microsphere whispering gallery mode system” Phys. Rev. Lett. July 2000, pp. 74-77, Vol. 85, No. 74 and also Knight, J. C., Cheung, G., Jacques, F., and Birks, T. A. “Phase-matched excitation of whispering-gallery-mode resonances by a fiber taper,” Opt. Lett., August 1997, pp. 1129-1131, Vol. 22, No. 15.) The resonances in these systems correspond to light trapped in circling orbits which can be used to minimize energy leakage (i.e. reflection).

Other techniques for minimizing energy loss from a solar cell formed from or coated with the nanoparticle dispersions described herein include: (i) adding an insulating layer; (ii) adding a back reflector, and (iii) patterning the surface or layer interfaces. For the creation of an insulating layer, oxidation can be used to add an encapsulating capping layer (i.e. SiO₂) to the nanoparticle substrate. For the back reflector, a metal such as aluminum can be used to trap energy. (See B. Sopori, W. Chen and Y. Zhang. “Development of a Thin Film Crystalline Silicon Solar Cell”. Presented at the National Center for Photovoltaics Program Review Meeting in Denver, Colo., Sep. 8-11, 1998. National Renewable Energy Laboratory (NREL), U.S. Dept. of Energy, November 1998.) For surface or interface patterning, the objective is to create a repetitive cycle of internal reflection or refraction that prevents energy from leaving the host material. Any design that facilitates this without significantly interfering with the optical and magnetic properties of the device is suitable. Using surface and interface patterns incident energy is continuously recycled within the host material and sent back for subsequent rounds of being converted (i.e. to useable visible or electrical energy) until, like the momentum in a pendulum, it is eventually depleted (i.e. converted with minimal to no absorption or external reflection).

With the energy conversion possibilities created by controllable, tunable, dopable nanomaterial dispersions and patterned substrates the term “solar cell” is really just a subset of potential applications or else a misnomer. In some embodiments, the coatings formulated in accordance with the teachings of the present invention would be better described as “thermal cells”. Categorically, the coatings of the present invention are better described as “energy translators”.

In addition to coating existing energy-harvesting (i.e. solar) cells with nano-dispersions, new cells can be formulated from raw materials (i.e. polymers) with nanoparticles directly incorporated therein. For example, titanium oxide (TiO₂) nanoparticles well dispersed in a solvent can be obtained and employed in a polymer matrix to make highly efficient dye-sensitized solar cells. By selectively choosing the polymers, this manufacturing approach can lead to more flexible, foldable and portable solar cell panels. The perfect spherical shape of the particles provide the largest surface area to make the translation more effective. These energy-harvesting, energy-translating nanopolymers (nanoparticles dispersed in a polymeric matrix) can be used in all of the traditional applications of polymeric materials such as for carrying cases for portable, rechargeable electronic devices.

In several embodiments the nanomaterials show magneto-optical properties, whereby the luminescence characteristics change in the presence of a magnetic field. Thus, the same material can provide different levels of security (from overt to covert) and the unique combination of properties can enable unbreakable security.

The useful functionalities of these nanoparticles that make them attractive for applications are provided primarily by their optical and magnetic properties. However, their metallic, conducting, semiconducting, and/or insulating, etc. properties can also be important for certain applications. In a preferred embodiment, the present invention emphasizes uniquely designed nanomaterials with nanoparticles that are active magnetically as well as optically. The present invention also emphasizes preferred methods for the synthesis of such nanoparticles and nanomaterials.

The tunability of the optical and magnetic characteristics of a set of nanoparticles to produce an array of unique sets of characteristics makes them suitable for many uses. For example, tunability makes nanostructures adaptable for optical fiber applications that require particular wavelengths. Tunability also makes nanostructures suitable for electrical components in regulated industries subject to numerous standards for safety and inter-compatibility.

The various functionalities of individual nanoparticles can be combined to form multi-functional composite structures (omnipotent nanomaterials) capable of light absorption and emission at a plurality of wavelengths in an “all in one” or “all from one” approach. A substrate for nanoparticles can be intentionally designed with a facet pattern that translates into functional units by utilizing reactivity differences throughout the pattern. A facet pattern can be controlled by selecting a suitable sample (i.e. index, misorientation direction and angle) and annealing conditions.

The teachings of the present invention enable tailoring nanoparticles with specific functionalities for particular applications. The nanoparticles of the present invention find uses in medicine, electronics, batteries (including rechargeable), energy generation, energy conversion (i.e. solar, thermal, UV, IR, visible), fiber optics, sensor devices, catalysts, photonics devices, high density magnetic recording components, recording media, color filters, dyes, optical filters, hair coloring products, flame retardants, corrosion protection coatings, photocatalysis, nonlinear optics, electroluminescent displays, photoluminescent sensors, biological probes, light-emitting quantum dots, quantum dot lasers, etc.

### BRIEF DESCRIPTION OF THE SEVERAL VIEWS OF THE DRAWING

FIG. 1 represents a single nanoparticle emitting at more than one wavelength, showing the photon energy dependent emission profile. The emission intensity at each wavelength depends upon the stimulation energy wavelength.

FIG. 2 represents a nanoparticle exhibiting magnetic, optical and magneto-optical properties useful for applications such as secure printing. The overt luminescence properties change to provide a separate covert pattern upon the application of a magnetic field with a threshold magnitude.

FIG. 3 represents a single nanoparticle, having blue, green and red emitting dopants, emitting white light as a result of their collective influence.

FIGS. 4A, 4B and 4C represent biomedical applications of functionalized nanoparticles (FNP).

FIG. 4A represents a functionalized nanoparticle (FNP) being attached to the surface of a cell (superficial cell labeling).

FIG. 4B represents a functionalized nanoparticle (FNP) being injected inside a cell (internal cell labeling).

FIG. 4C represents a functionalized nanoparticle (FNP) with magnetic properties being used to label a drug molecule (DM) in order that, upon application of an external magnetic field, the drug molecule will be carried to a target site.

FIG. 5 represents a multifunctional nanoparticle coating on a solar cell. Different dopants in the nanoparticle coating can be used to enable it to convert several different forms of energy to visible light, which together with its antireflective qualities, enhances solar cell efficiency by increasing the effective amount of visible light available for subsequent conversion to electrical energy.

FIG. 6A illustrates controlled doping and fabrication of nanoparticles with tailored emission features. The photoluminescence (PL) spectra from alumina is shown along with those after doping it with Fe, Cr, and Zn. Each of these dopants gives rise to characteristic emission and the spectrum after doping with the three dopants is a broad emission covering the range 630-850 nm (the top curve in A).

FIG. 6B displays a demonstration of the selective filtering process in order to have the desired component. The arrows indicate the main wavelength that is filtered out. The intensity values are the actual intensity and therefore clearly illustrate the tunability of the emission.

### DETAILED DESCRIPTION OF THE INVENTION

The invention is centered around the controlled synthesis of nanoparticles (NP) (preferably oxide based particles) through a combination of sol-gel processes, sonochemistry and doping. Both metal (M1) and bi-metal (M1, M2) isopropoxide sol-gels can be further processed with sonication and doping (D) to generate spherical shaped luminescent NP. Optionally, the solutions may also be annealed to encourage the proliferation of nano-scale structures. Also optional, is re-sonication of the solutions to create greater uniformity of nanoparticle size distribution.

The following symbology represents the various oxide nanostructure possibilities:

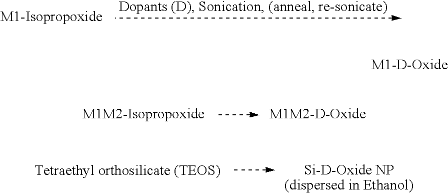

This approach can be generalized and applied to other systems (including non-oxide systems) as well. The use of bimetallic precursors and a greater array of non-toxic soluble dopants opens up enormous possibilities. When applied to security applications (i.e. ink and toner dispersions), this approach enables an infinite number of codes through the judicious combination and alteration of variables such as: (i) host materials, (ii) dopants (number, concentration, wavelengths, etc.), (iii) synthesis parameters (particle size and shape) and (iv) synthesis conditions (sonication intensity/rate/duration, annealing temperature/duration, etc.). Thus, the same host material can be used as a matrix for distributed NP with a wide range of properties and multiple functionalities depending upon numerous input variables (i.e. wavelength of light used for stimulation; existence, direction, and strength of a magnetic field, etc.). For example, largely iron doped alumina is a black material which emits red light upon shining with ultraviolet light and emits near IR light upon shining with green light.

In a preferred embodiment of the present invention, omnifarious oxide nanoparticles that emit brightly at multiple wavelengths are synthesized. Different types of oxide sols can be made using different precursors. For example, aluminum isopropoxide can be used as a precursor for an alumina oxide sol and tetraethoxy orthosilane (TEOS) can be used as a precursor for a silica oxide sol. The sol is prepared by sonicating the isopropoxide in water. The mixture is sonicated at bearable warmth and a clear solution is formed. This is followed by the addition of the dopant source, preferably in a salt form soluble in water. The doped mixture is sonicated thoroughly, transferred into a crucible, and (optionally) annealed at a temperature at or above the decomposition temperature of the oxide. After annealing, the resulting material is found to be in the nanoparticle form. The particles range in size from 5-100 nm and spherical shapes predominate. At about the 10 nm or less size scale, nanoparticles are also called “quantum dots”. The size of the particles can be tuned by optimizing the sonication conditions. Uniformly sized particles can be obtained by re-sonicating the annealed material.

The advantage of this manufacturing approach is that the light emitting dopant ions (of one or more variety) are readily available for incorporation into the host lattice during its formation. Therefore, it is possible to control incorporation of the desired ions to achieve concentration levels substantial enough to be effective with a noticeable alteration of the optical and magnetic properties of the host matrix material. The dopants can be made more effective by electrochemical treatment, which results in well separated cations and anions.

Using the processes of the present invention both organic and inorganic materials can be used to create unique nanomaterial dispersions and nanoparticulate coated or embedded substrate designs with particular functionalities. Inorganic materials including both semi-conductors (i.e. silicon, germanium, gallium and indium) and metallic conductors (i.e. iron, cadmium) can be used in their pure form or as part of compounds (i.e. gallium arsenide, indium phosphate, iron oxide, cadmium sulfide, etc.).

Organic materials including collagen, fibrinogen, etc. may be used as a host matrix or added to a sol-gel mixture prior to the “nano-processing” steps of doping, sonicating, annealing (optional), and re-sonicating (optional). Carbon nanotubes and fullerenes can be coated with one or more type of and/or one or more size of photosensitive nanoparticle (quantum dot). Organic molecules including metal-thalocyanines and aryl amines can be used in a hole conducting solution in which the nanoparticles are dispersed or in a hole conducting layer adjacent to a nanoparticle layer in a device. (See U.S. Published Application No. 2008/0066802 at [0055].) Some types of organic molecules (i.e. organic semiconductors, polyphenylene vinylene, copper phthalocyanine, and carbon fullerenes) can be combined with nanoparticles in solar cell devices. Although the energy conducting and conversion efficiencies for organic materials are generally lower than those for their inorganic counterparts, organic materials can nevertheless be valuable where biocompatibility, mechanical flexibility and disposability (including bioabsorbability, biodegradability and bioerodibility) are important. Unlike conventional solar cell devices that depend on the electric field generated by a p-n junction to separate electrons and holes, in organic solar cells the electron-hole pairs typically remain bound as an exciton. Organic cells generally have an electron donor material and an acceptor material. Typically, the photon is converted into an exciton in the donor layer and the electron-hole pair (exciton) remain bound until the exciton reaches (i.e. via diffusion) the donor-acceptor interface. Short exciton diffusion lengths tend to limit efficiency but nanostructured interfaces appear to improve performance. Other organic materials (fluorescent small molecules, polymers and phosphorescent materials) have been applied in light emitting displays (OLEDS) for electronic devices. (See U.S. Published Application No. 2007/0194694 at paragraphs [0003]-[0007].)

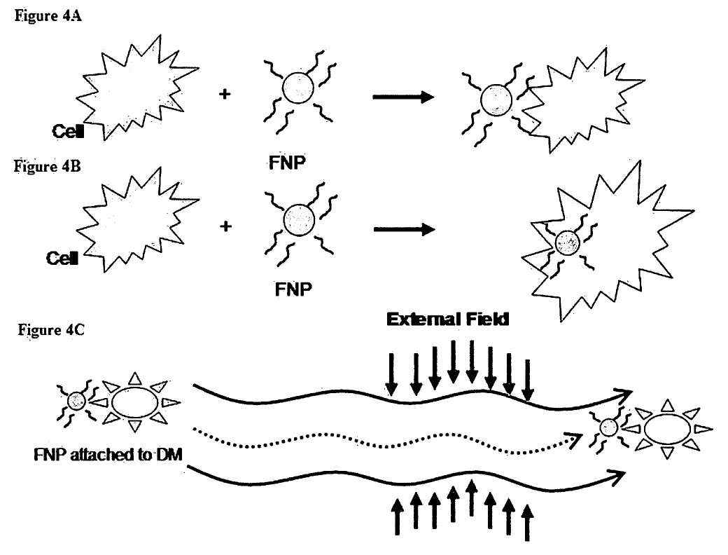

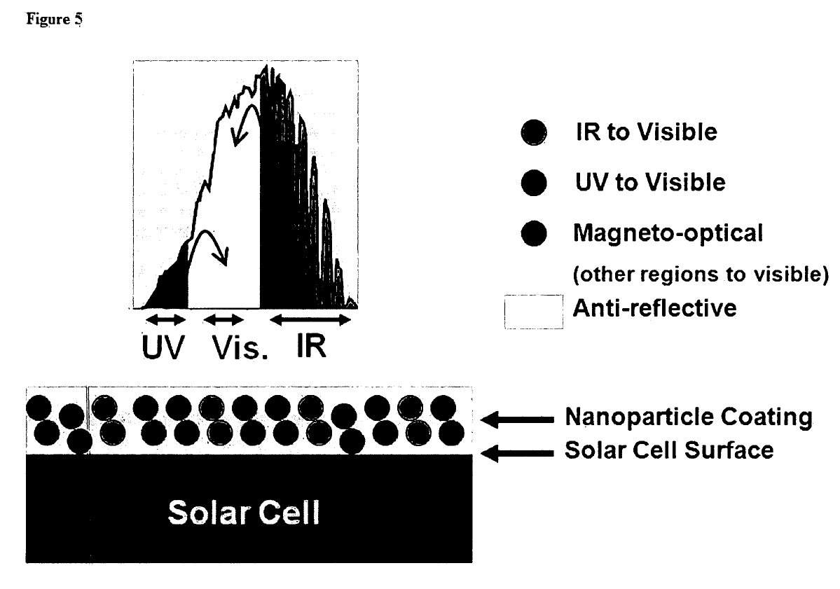

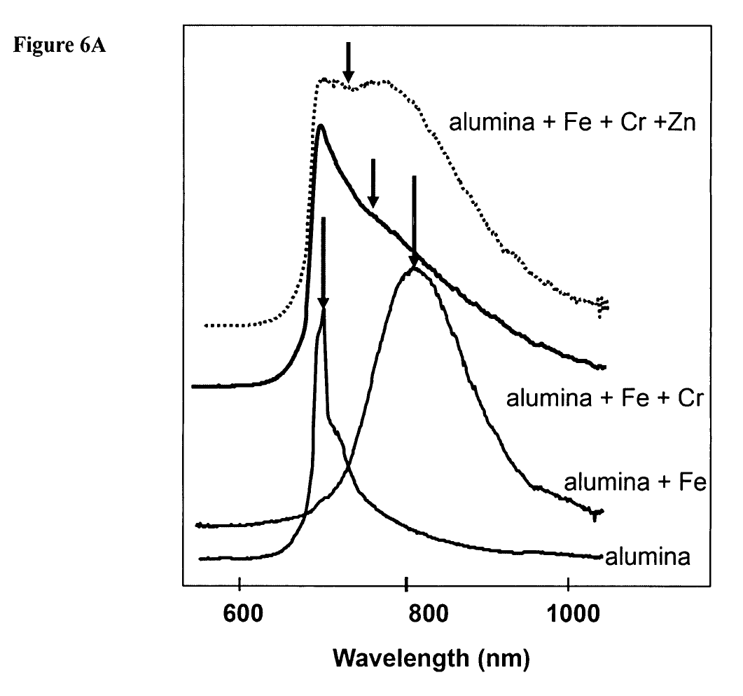

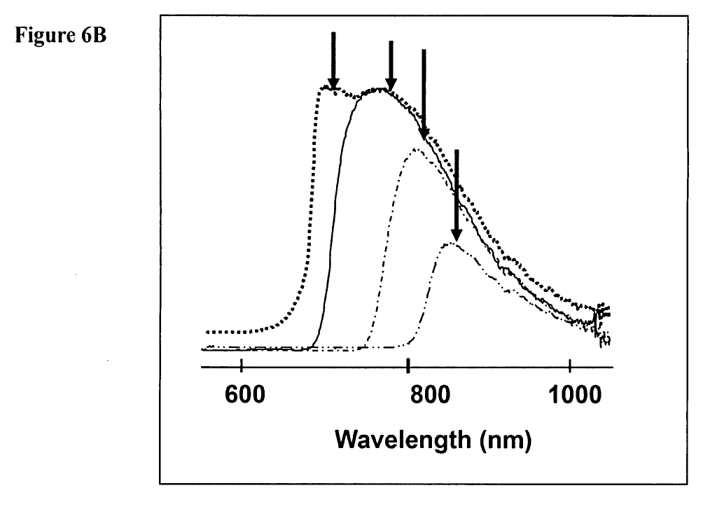

Functionalized nanoparticles can be further functionalized to terminate with organic groups (i.e. carboxylic acid groups, phosphonic acid groups, sulfonic acid groups, amine containing groups, etc.) for attachment and labeling of cells. Linker molecules can be used in which one end reacts with the organic group on the functionalized nanoparticle while the other end of the linker reacts with a reactive site on the target cell. Linker molecules have also been known to have other benefits including: passivating nanoparticles (NPs); increasing stability, light absorption and photoluminescence; and enhancing solubility in some organic solvents. (See U.S. Published Application No. 2008/0066802 at [0053].) Longer linker molecules, known as “spacers” (i.e. carbon spacers between 6 to 20 carbon atoms) may also be used to prevent steric hindrance during the interaction between the reactive group on the target molecule and the reactive group on the functional nanoparticle (or on its spacer). (See U.S. Pat. No. 6,514,481 at 3:2-6 and 3:22-28.) In addition, nanoparticles can be coated with organic materials to disguise them for insertion within a cell. (See FIGS. 4(A), 4(B) and 4(C).)

According to one embodiment, iron oxide nanoparticles are formed on a silicon substrate. Silicon wafers with as-incorporated amorphous iron oxide nanoparticles exhibit superparamagnetic behavior but after annealing the same samples show ferromagnetic property attributed to transformation of the amorphous iron oxide into crystalline nanoparticles of Fe. Upon annealing, experimental results clearly demonstrate that Fe₂O₃ particles are reduced to elemental Fe. The reduction temperature of iron oxide on a semiconductor substrate is dictated by the temperature at which the semiconductor element oxide desorbs. (See K. Prabhakaran et al. “Nanoparticle-Induced Light Emission from Multi-Functionalized Silicon”. Advanced Materials; Vol. 13, No. 24, pp. 1859-1862. Wiley-VCH, Dec. 17, 2001.) Light emission intensity spectra as a function of sample temperature suggests that the process is thermally activated and that the origin is exciton related.

According to another embodiment a semiconducting silicide such as β-FeSi₂ is used as the host substrate for nanoparticle bottom-up derivation. The use of β-FeSi₂ is of special interest because it is covalent and environmentally friendly with a direct bandgap. It shows potential for use as a silicon-based light emitter. More specifically, β-FeSi₂ appears especially useful for fiber optic communications because of the wavelength(s) of light it emits.

Cadmium sulfide (CdS) has been shown to self-organize into ring structures (including the wurtzite (hexagonal structure)) that exhibit luminescent properties. The formation of ring structures appears to result from a drying process of microscopic droplets containing the particles which nucleate on the silicon oxide surface during the ultrasonic treatment. The instability of surface films on silicon substrates creates surface ripples when the films rupture. Droplets nucleate on these regions. In one embodiment, rings of CdS have been found to form when placed in an ethanol suspension upon a silicon surface having a thin oxide coat and annealed in an ultrahigh vacuum chamber (UHVC) above the silicon-oxide decomposition temperature (>800° C.). (See K. Prabhakaran et al. “Luminescent Nanoring Structures on Silicon”. Advanced Materials; Vol. 15, No. 18, pp. 1522-1526. Wiley-VCH, Sep. 16, 2003.) When the particles are well separated in ring formations (i.e. after annealing), the spectroscopy signals are reduced despite the presence of a large number of particles. The exact pattern formed by the ring structures is a product of the combined influence of several factors including: surface tension, viscosity, interparticle interaction, particle-surface bonding and substrate heterogeneity. Accordingly, one or more of these variables can be modified to tune the nanostructure for a particular application (i.e. nano-electronics). The tunability of nanostructures and the array of variables to manipulate provide endless possibilities for specific applications.

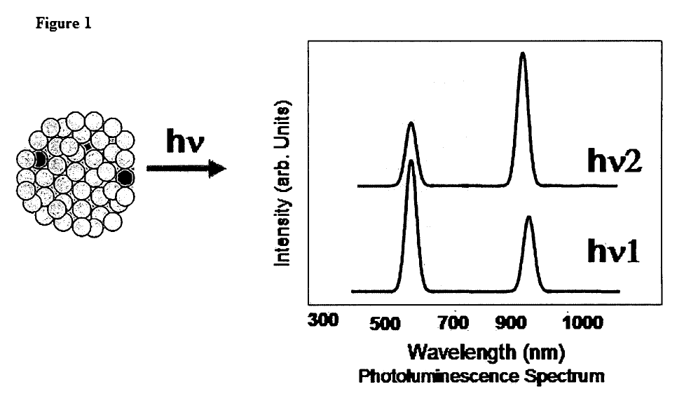

FIG. 1 shows the emission spectra from a NP doped with two different ions. The emission profile shows that the emission spectra depends upon and changes with the excitation energy. In this case there is a reversal in the intensity of the emission lines around 550 nm and 900 nm when the excitation energy changes from hv₁ to hv₂. This phenomenon can be effectively utilized for security coding and for bio-applications.

In security coding, genuine records, disks, or product labels would show the unique pattern of emission dependent on stimulation wavelength. Counterfeit goods and pirated trade labels, in contrast, would not show the unique and variable (stimulation energy dependent) emission profile of their authentic counterparts. Sophisticated copycats may be able to duplicate a single emission profile (i.e. at a single energy stimulation wavelength) but by increasing the number and variability of dopants used in the nanomaterial dispersions, a multi-tiered complex code that is impossible to reverse engineer can be created.

In bio-applications the stimulation dependent emission spectra can be exploited in multi-stage therapeutic treatment regimes. First, target cells are labeled with nanoparticles. Target cells can be labeled on the surface or modified internally. Surface NP labels can be accomplished via coatings, bonding, attachment, ionic interactions, etc. directly or indirectly via linker or spacer molecules. Cells can be modified internally by injection, phagocytosis of the NP by the cell, NP diffusion across cell membranes, or NP transport through cell channels. Once the target cells are made distinguishable from non-target cells, a first cycle of treatment can be initiated by stimulating the region with a first energy or magnetic field. After a period of time a second cycle of treatment can be initiated by stimulating the region with a second energy or magnetic field. The process continues until the target cells have been effectively treated. The treatment cycle could also alternate back and forth or repeatedly move through a sequence of stimulation energies to separate periods of more intense treatment (determined by the impact of the emission wavelength and intensity) with periods of more mild treatment.

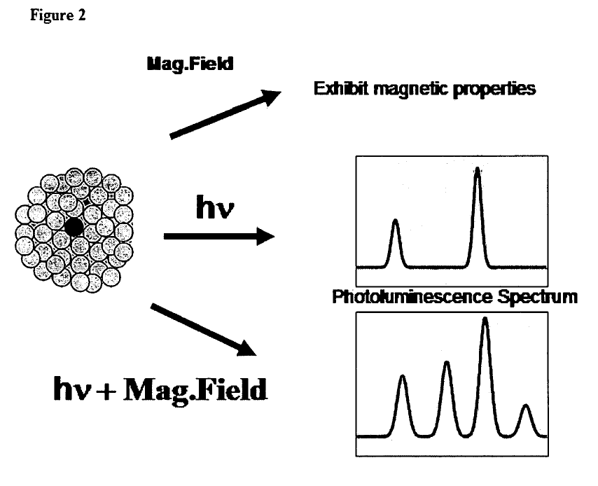

FIG. 2 depicts the case where the addition of one or more magnetically active ion into the host lattice induces the occurrence of unique emission lines upon the application of a magnetic field to the nanomaterial. This magnetically active material can be used to enhance security by providing additional discriminatory features to differentiate counterfeits or copies from authentic, certified, or licensed products. Counterfeit producers may find it more difficult to become aware of and to replicate an authentic label's magnetic sensitivity. Magnetically active nanomaterials can also be used for bio-applications, where an external magnetic field can be used to induce light emission. For example, there is evidence that the emission of light of certain wavelengths can be useful for preventing intimal hyperplasia in the treatment of clogged arteries. (See Kohyama, S., et al. “Effectiveness of Narrow-Band Ultraviolet-B Phototherapy for Prevention of Intimal Hyperplasia in a Rat Carotid Balloon Injury Model”. Lasers in Surgery and Medicine; Vol. 39, pp. 659-666. Published online by Wiley-Liss, Inc., 2007.)

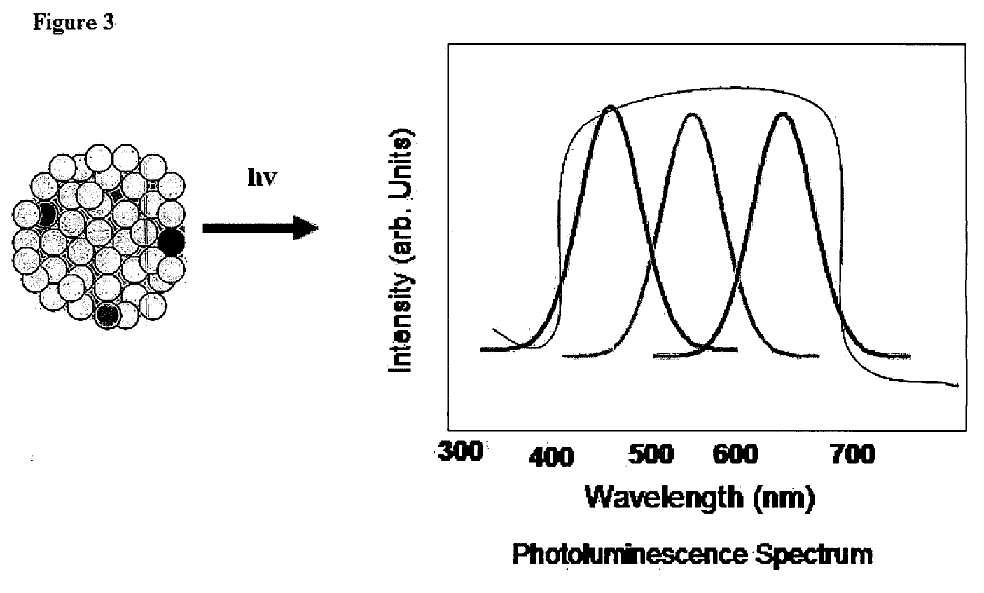

FIG. 3 shows the emission of white light by a combination of red, blue and green emitting ions incorporated into the same nanoparticle. Thus, an individual nanoparticle with multiple variable dopants can be expected to produce strong, coherent emission spectra. Additionally, a broad emission spectra characteristic of white light can also be generated from the singly doped NP due to crystal field effects. For example, silica NPs sonochemically synthesized from TEOS have been shown to emit intense white light.

FIGS. 4(A), 4(B), and 4(C) illustrate a few of the possible biomedical applications of these unique materials. FIG. 4(A) shows a spherical NP that has been functionalized (FNP) to terminate with reactive groups (i.e. —NH or —COOH groups) so it can be attached to bio-cells and used as a label and as cellular recognition material. FIG. 4(B) illustrates the possibility of inserting the NP into a live cell (i.e. via diffusion, channel transport, phagocytosis of the NP, etc.). The optical properties (or other properties) of the NP can be monitored from its position inside the cell where it rapidly responds to changes in a host cell's chemical environment. Thus, the NP can function as a “cellular policeman” by alerting scientists and physicians to changes in cellular chemistry at a very early stage. In this manner, NPs can detect and signal the occurrence of undesirable events such as invasion of the cell. These “cellular policeman” NPs can be inserted within even healthy cells adjacent a cancerous or infected site to monitor the spread of a disease and identify proliferation promptly enough for a better chance of controlling it.

The light emission property of the NPs attached to or inserted within bio-cells can distinguish the cell from a group of other cells. Other properties of NPs can also be used in this manner (i.e. magnetic, metallic, insulating, semiconducting, conducting, etc. properties). Through the distinctiveness of cells associated with NPs, one can investigate the onset mechanism of deadly diseases such as cancer at a cellular and sub-cellular level.

As shown in FIG. 4(C), NPs containing one or more magnetically active ion provide a new method for targeted drug delivery. The magnetically functionalized NP (FNP) can be attached to a drug molecule (DM) and the trajectory of the drug can be controlled (direction and speed) by application of an external field (shown by red arrows). In this manner, the drug can be directed to a desired location for action. This targeted drug delivery approach avoids the drawbacks of sloppy systematic, regional, or even local (but not target) delivery methods in which the drug's domain is both overbroad (in that it unnecessarily impacts healthy cells) and too narrow (in that it does not adequately impact diseased cells, i.e. because of concentrations that are too low due to safety concerns).

The ability of nanostructures to self-assemble permits their self-endowment with unique functions and qualities upon formulation before being integrated into larger systems with other components. This increases the stand-alone value of nanostructures. One application for stand-alone nanostructures is incorporation upon semiconductor substrates. The idea of incorporating externally synthesized nanoparticles onto semiconductors has been termed a “plug and play” approach to the multi-functionalization of silicon. (See K. Prabhakaran et al. “Nanoparticle-induced multi-functionalization of silicon: A plug and play approach”. Applied Surface Science; Vol. 190, pp. 161-165. Elsevier Science B.V., 2002.) This semiconductor fabrication method is also referred to as a “bottom-up” approach and can be combined with spintronics for the production of cutting-edge nanoelectronic devices.

A preferred embodiment of nanostructure semiconductors is the bottom-up formulation of β-FeSi₂. Silicon is particularly well suited as a substrate for nanostructures because its atomic steps: (i) have high reactivity, (ii) exhibit excellent affinity for adsorbing foreign species, and (iii) act as nucleation centers for further growth. However, other non-silicon or non-pure silicon (i.e. silicon compound) materials can also be used as nanostructure substrates provided they do not impair the unique functionalities (i.e. luminescence, optical, magnetic, metallic, conducting, semiconducting, insulating, etc.) of nanoparticles.

To further increase the functional possibilities for nanomaterial semiconductors, the step sizes and edges of the substrate surface can be manipulated via means such as traditional etching. When annealing is used as part of the nanostructure formulation process, the particles remaining after annealing tend to be of uniform size and to nucleate preferentially at surface step edges. The size distribution of the NPs deposited or formed on a substrate surface tends to be narrow because when the NP suspension is prepared (i.e. NPs suspended in ethanol) the larger particles sediment out of solution early on. This uniformity is advantageous for ensuring predictable and homogenous properties throughout the substrate.

Intentionally etched semiconductor surfaces can also be used to direct the assembly of nitride linings. The linings form from bifunctional nitric oxide during nitridation reactions at elevated temperatures. Nitric oxide is bifunctional in that both the nitrogen and oxygen species are reactive when the molecule breaks down (i.e. on a silicon substrate at high temperatures). Oxygen etches silicon while nitride deposits itself in particular patterns corresponding to the locations etched by oxygen. Through the dissociative adsorption of nitric oxide from a substrate, reactive oxygen becomes available to etch the substrate. Oxygen atoms generate reactive centers by forming dangling bonds and unsaturated bonds on silicon. Nitrogen atoms respond by becoming attached at these same positions. The combined etching processes of step band formation and reactive center generation produce a pattern that precedes and serves as a template for the deposition of nitride linings. (See K. Prabhakaran, et al. “Ultrafine and Well-defined Patterns on Silicon Through Reaction Selectivity”. Advanced Materials; Vol. 14, No. 19, pp. 1418-1421. Wiley-VCH, Oct. 2, 2002.) Nanoparticle dispersions can then be deposited upon the nitride linings.

Nanostructures can be designed to intake different sources and forms of energy as stimulation depending on the application. For example, some nanostructure embodiments may be stimulated by lasers (i.e. He—Ne or Ar) while other embodiments depend upon ultraviolet (UV), infrared (IR), or visible light. In addition, some nanostructure embodiments may be stimulated by non-light energy sources (i.e. radiofrequency waves (RF), microwaves, etc.). Some omnipotent or multifunctional nanomaterials (i.e. with a variety of dopant compositions or sizes) absorb and react to more than one source and form of energy for stimulation. Raising the temperature (i.e. during the annealing process or as part of the stimulation process) and/or stimulating the nanostructure surface with light beams both have been shown to diminish the spectroscopy signals by inducing desorption of excess NPs and/or bombarding NPs from a surface. NPs that absorb energy at lower temperatures and or from sources other than light beams could prevent these losses. Alternatively, an insulating layer above the NP layer can reduce surface displacement losses.

Similarly, nanostructures can be designed to output different sources and forms of energy as emission depending upon the application. In solar cell applications one desired energy output form is visible light which can be produced by the nanomaterials from both IR and UV forms. The solar cell then uses the visible light (direct and indirect from IR, UV, etc.) to make electrical energy.

The nanoparticles of the present invention can be applied in medical applications including providing pinpoint lighting in biodiagnostic probes precisely at a target site. The nanoparticles can also be used to distinguish certain cells requiring treatment (i.e. malignant cells) from others via superficial attachment or internal labeling (see FIGS. 4(A), 4(B) and 4(C)). Treatment options that reach the NP cells exclusively, can then be used to provide more intense and more efficient therapy that does not unnecessarily weaken healthy cells.

Alternatively, NPs can also be used on the other side of the reaction, applied to the drug molecules or other external treatment agents rather than internal cells. Incorporating NPs within therapeautic agents can produce formulations that will only react with afflicted target cells. In one embodiment, NPs can be included within coatings on therapeautic agents (i.e. molecules, drugs, capsules, etc.) so that the agents are only attractive to (absorbed by) and reactive with select cell types (i.e. afflicted target cells).

In the electronics field, the technology of the present invention is especially advantageous for mobile personal electronics. Although the mobility of electronics has come a long way, professionals are still restrained by the continual need to find an electric outlet to recharge. This interferes with productivity and impairs flexibility and freedom. Many popular public working sites (i.e. coffee houses and airports) do not have one outlet per person and people must hunt for electrical outlets and stretch cords across walking spaces creating a tripping hazard. Further, with the increasing popularity of working on-the-go such public sites are likely suffering a substantial increase in their energy bill by customers and non-customers alike that continually recharge or plug-in to free power.

The NPs dispersions of the present invention, with their ability to harvest and transform light and energy, can provide an alternative that will benefit everyone. By attracting and trapping ambient room energy (i.e. including inside artificial light and heat), FNPs can create energy compatible with mobile personal electronic devices. Other contemporary non-electrical power alternatives are weaker because they require intense natural solar energy and charging periods that cannot keep up with the power depletion rates of ordinary users (i.e. with habits including simultaneously running several programs, downloading large files, long working sessions, etc.) Thus, with the present invention, professionals need not live in Phoenix (or another site of dependable sunshine) to recoup the benefits of their investment in new energy technologies. Further, professionals whose work requires electronic devices can work from a much greater array of places without increasing the energy bill of others (i.e. coffee house owners, municipal libraries, etc.) when working off-site. In some embodiments, to economize on device size the display surface could also function to capture energy.

The magnetic properties of the FNPs of the present invention may also be tailored for use in electronic device memories. FNPs can create physically smaller internal memories with more storage space and faster access and retrieval.

From the above description of the invention, those skilled in the art will perceive improvements, changes and modifications. Such improvements, changes and modifications within the ordinary skill of the art are regarded as covered by the appended claims directly or as equivalents.

What is claimed is:

1. A dispersion of nanoparticles in a solvent, further comprising one or more dopants, wherein the doped nanoparticle dispersion is functional in that it can absorb energy of at least one form and convert it to energy of at least one different form.

2. The dispersion of claim 1, wherein infrared energy can be absorbed and converted to visible light.

3. The dispersion of claim 2, produced by sonicating a semiconductor precursor with a rare earth salt.

4. The dispersion of claim 3, wherein the semiconductor precursor is tetraethyl orthosilicate (TEOS) and the rare earth sale is lutetium nitrate (LuNO₃).

5. The dispersion of claim 2, wherein ultraviolet energy can also be absorbed and converted to visible light and the dispersion is antireflective.

6. The dispersion of claim 1, wherein at least one dopant is magnetic.

7. The dispersion of claim 6, wherein the magnetic dopant is selected from the group consisting of: an iron (Fe) ion, a chromium (Cr) ion, and a copper (Cu) ion.

8. The dispersion of claim 6, wherein upon the application of a magnetic field to the dispersion, an emission intensity for at least one form (i.e. wavelength) of energy is different than its emission intensity without the magnetic field.

9. The dispersion of claim 1, wherein at least one nanoparticle is selected from the group consisting of: silicon oxide (SiO₂), titanium oxide (TiO₂), and aluminum oxide (Al₂O₃).

10. The dispersion of claim 1, wherein at least one nanoparticle is a metal oxide.

11. The dispersion of claim 10, wherein the nanoparticles comprise at least two different types of metal oxides, thereby making the dispersion at least bimetallic.

12. The dispersion of claim 1, wherein the solvent is ethanol and at least one dopant is selected from the group consisting of: Eu, Fe, Zn, F, Cr, Co, Cu, Sn, Li, K, Mg, Mn, and Ce.

13. The dispersion of claim 1, wherein the nanoparticles and the one or more dopant are uniformly distributed within the solvent.

14. The dispersion of claim 1, wherein the nanoparticles are spherical and of uniform size.

15. The dispersion of claim 1, wherein the nanoparticles form ring structures when the dispersion solidifies.

16. The dispersion of claim 1, further comprising a photovoltaic or solar cell, wherein the doped nanoparticle dispersion is either incorporated within or coated upon the cell.

17. The dispersion of claim 1, further comprising a substrate, wherein the doped nanoparticle dispersion is applied to the substrate for use in electronic applications.

18. The dispersion of claim 1, further comprising one or more target biological cell within a body, wherein the dispersion is attached to a surface of the cell or inserted within the cell.

19. A method of producing a nanoparticle dispersion comprising:
(i) dissolving an isopropoxide sol-gel or tetraethyl orthosilicate (TEOS) in a solvent to form a solution;
(ii) adding a dopant to the solution;
(iii) sonicating the solution; and
(iv) optionally, adding more dopant to the solution; and
(v) optionally, re-sonicating the solution.

20. The method of claim 19, further comprising the step(s) of:
(iv) annealing the solution; and
(v) optionally, re-sonicating the solution.
